(19) United States
(12) Patent Application Publication
(10) Pub. No.: US 2026/0154937 A1
(43) Pub. Date: Jun. 4, 2026

(54) Image Processing for Surgical Applications

(71) Applicant: Leica Instruments (Singapore) Pte. Ltd., Singapore (SG)

(72) Inventors: Milos SORMAZ, Singapore (SG); Patrick SZABO, Singapore (SG)

(21) Appl. No.: 18/619,225

(22) PCT Filed: Sep. 29, 2022
(86) PCT No.: PCT/EP2022/077168

§ 371 Date: Mar. 28, 2024

(30) Foreign Application Priority Data
Sep. 30, 2021 (DE) 10 2021 125 412.4

(51) Int. Cl. G06V 10/75; A61B 90/25; G06V 10/44

(52) U.S. Cl. CPC G06V 10/751; G06V 10/44; A61B 90/25; G06V 2201/03

(57) ABSTRACT

An image processing device is disclosed. The device selects an intra-op image which is stored in a memory. The intra-op image has a field of view. The device determines a matching image based on: selecting the matching image from the memory, or constructing the matching image from image data. The matching image matches the field of view of the intra-op image.

### TECHNICAL FIELD

Examples disclosed herein relate to image processing for surgical applications.

### BACKGROUND

Modern surgery can be performed using information from multiple imaging modalities. For example, intraoperative imaging can include surgical optical microscopes, ultrasound, and other modalities. A surgeon may utilize information from pre-operative images as well. After surgery, post-operative images may be taken. Many challenges arise when dealing with multiple sources of images which may be taken at different times (e.g. pre-, intra-, and post-op images).

### SUMMARY

It may be desirable to improve the ability of healthcare workers, students, and researchers to access and compare images related to patient care.

Herein is disclosed an image processing device as defined in claim 1, a surgical imaging device as defined in claim 11, a method of image processing as defined in claim 13, and a computer program as defined in claim 22.

In an embodiment, an image processing device is configured for: selecting an intra-op image which is stored in a memory. The intra-op image has a field of view. The image processing device determines a matching image based on: selecting the matching image from the memory, or constructing the matching image from image data. The matching image matches the field of view of the intra-op image. Providing matching fields of view of images to medical professionals can aid in simplifying the interpretation of the images.

In an embodiment, the image processing device selects the intra-op image from a plurality of intra-op images stored in the memory. Providing matching fields of view of images to medical professionals can aid in the interpretation of the images. It can reduce computational complexity to have the matching fields of view already in memory.

In an embodiment, the image processing device determines a second matching image. The second matching image matches the field of view of the intra-op image. The matching image can be a pre-op image, and the second matching image can be a post-op image. Providing matching fields of view of images to medical professionals can aid in the interpretation of the images. Providing them for images which are acquired at different times can simplify the image analysis and save the time of the medical practitioner.

In an embodiment, the intra-op image is an image captured by a surgical imaging apparatus. Medical practitioners can work more efficiently and have easier intuitive understanding of the images when they have matching fields of view. Providing such images, when the images are acquired at different times and/or with different apparatuses can be burdensome. Here, the providing of the images can be simplified for the medical practitioner.

In an embodiment, the image processing device includes a surgical imaging apparatus which is a microscope, infrared microscope, optical coherence tomography device, photoacoustic imaging device, ultrasound imaging device, magnetic resonance imaging device, or computed tomography device. These modalities can be preferred means of acquiring images in a surgical environment.

In an embodiment, the image processing device selects a second intra-op image which is stored in memory. The second intra-op image matches the field of view of the intra-op image. Optionally, the second intra-op image is automatically selected when the intra-op image is selected. Providing images with matching fields of view can aid in the comparison of surgical outcomes. The providing of the images can be simplified for the medical practitioner.

In an embodiment, the second intra-op image is captured at a plurality of acquisition parameters, and the first intra-op image is captured at the plurality of acquisition parameters. It is possible that the acquisition parameters of the second intra-op image are determined automatically, e.g. by reading the acquisitions parameters of the first, which have been stored, e.g. with metadata of the first intra-op image. Providing images with matching fields of view can aid in the comparison of surgical outcomes.

In an embodiment, the image data includes three dimensional imaging data. The image processing device can construct the matching image by determining a plane of cross-section of the three dimensional imaging data based on a plurality of stored parameters associated with the intra-op image. The providing of the images of matching fields of view can be simplified for the medical practitioner.

In an embodiment, the stored parameters includes at least one of: a user input, a position of a detector, an orientation of the detector, a plurality of reference positions, a magnification, a focal length, or a working distance. Such parameters can provide at least one way of determining the matching image such that it has a matching field of view.

In an embodiment, the image processing device can construct the matching image by an algorithm which includes edge recognition. Edge recognition can provide a way of matching the fields of view of the images, e.g. to aid the practitioner's interpretation of the image data.

In an embodiment, a surgical imaging device captures an intraop image and determines a matching image based on constructing the matching image from image data. The matching image matches the field of view of the intra-op image. The surgical imaging device stores the intraop image. The surgical imaging device stores the matching image and/or acquisition parameters for the determination of the matching image from the image data. Providing matching fields of view of images to medical professionals can aid in simplifying the interpretation of the images. For example, the acquisition parameters are stored in meta-data of the intraop image. Providing a means to match the fields of view of images provided to medical professionals can aid in simplifying the interpretation of the images. The surgical imaging device can include a detector for capturing the intra-op image. The surgical imaging device can include a processor (e.g. for controlling the device) and/or a memory (e.g. for storing/retrieving images).

In an embodiment, the surgical imaging device stores acquisition parameters of at least one of: working distance, magnification, focal length, position of the detector of the intraop image, orientation of the detector, coordinates of a cross-section of the image data which includes the matching field of view, or user input. Optionally, the surgical imaging device can include a sensor(s) and/or fiducial marker(s) for sensing/determining the position and/or orientation of the detector. Providing a means to match the fields of view of images provided to medical professionals can aid in simplifying the interpretation of the images.

Herein is disclosed a method of image processing, comprising selecting an intra-op image which is stored in memory. The intra-op image has a field of view. The method includes determining a matching image based on: selecting the matching image from the memory, or constructing the matching image from image data. The matching image matches the field of view of the intra-op image. Providing matching fields of view of images to medical professionals can aid in simplifying the interpretation of the images.

The intra-op image can be selected by a user. It is convenient for a user to be able to select the image which is then provided with a matching image from the image data.

The method can further include: determining a plurality of parameters during a surgery, capturing the intra-op image during the surgery, and storing the plurality of parameters in association with the intra-op image. The parameters can reduce the computational complexity of determining the matching image.

The stored parameters can include a position and/or an orientation (e.g. of the detector of the intraop image) for providing the field of view of the intra-op image. Having the position/orientation stored can reduce the computational complexity of determining the matching image.

Constructing the matching image can include recognizing edge features. Recognizing edge features can reduce the computational complexity of determining the matching image.

The matching image can be a pre-op image or a post-op image. Providing matching fields of view of pre- and/or post-op images to medical professionals can aid in simplifying the interpretation of the images.

The method can also include determining a second matching image which is a post-op image. The matching image can be a pre-op image. Providing matching fields of view of pre- and/or post-op images to medical professionals can aid in simplifying the interpretation of the images.

The method can also include selecting a second intraop image, and determining a second matching image (302) based on: selecting the matching image (302) from the memory, or constructing the matching image from image data (350). The matching image matches the field of view of the second intra-op image. Providing matching fields of view of multiple images to medical professionals can aid in simplifying the interpretation of the images.

Herein is disclosed, according to an embodiment, a computer program with a program code for performing the methods described herein.

### SHORT DESCRIPTION OF THE FIGURES

Some examples of apparatuses and/or methods will be described in the following by way of example, and with reference to the accompanying figures, in which

FIG. 1A illustrates a group of images according to an embodiment described herein;

FIG. 2A illustrates a group of intraop images according to an embodiment described herein;

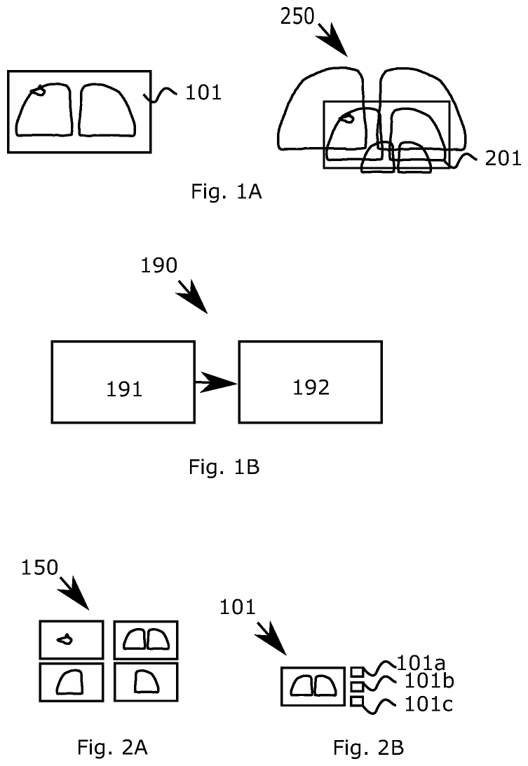

FIG. 1B illustrates a method of image processing according to an embodiment described herein;

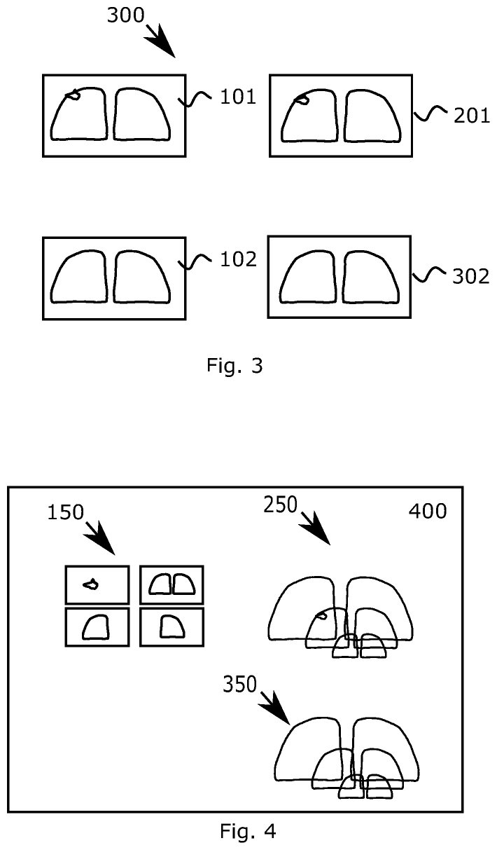

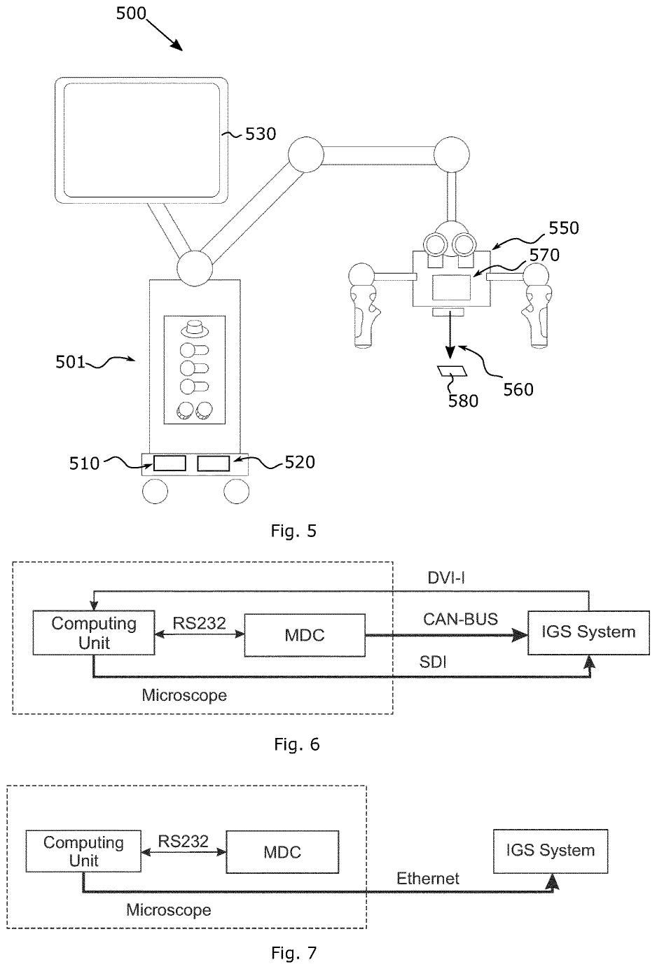

FIG. 2B shows an intraop image and associated parameters according to an embodiment described herein;

FIG. 3 illustrates a group of images according to an embodiment described herein;

FIG. 4 illustrates a memory device according to an embodiment described herein;

FIG. 5 illustrates a surgical imaging system according to an embodiment described herein;

FIG. 6 illustrates a block diagram of a surgical imaging system according to an embodiment described herein; and

FIG. 7 illustrates a block diagram of a surgical imaging system according to an embodiment described herein.

### DETAILED DESCRIPTION

Various examples will now be described more fully with reference to the accompanying drawings in which some examples are illustrated. In the figures, which are not to be assumed to be to scale, the thicknesses of lines, layers and/or regions may be exaggerated for clarity. As used herein, the term “and/or” includes any and all combinations of one or more of the associated listed items and may be abbreviated as “/”. Herein, a trailing “(s)” indicates one or more; for example remote device(s) indicates one or more remote devices.

Herein, intraoperative image, or intra-op (or intraop), can mean an image captured/acquired during the course of a surgical operation and/or while the patient is in a surgical suite for the surgical operation. Pre-operative images (pre-op images or preop images) can be those which are captured before the course of the surgical operation and/or before the patient is brought to the surgical suite. Post-operative images (post-op images or postop images) can be those which are captured/acquired after the surgical operation and/or after the patient is removed from the surgical suite.

Herein “image data” can be used interchangeably with an “image data set.” Image data can provide data for a three dimensional image, and/or can be data which can be processed to generate two dimensional images along various planes. For example, image data can be used to determine two dimensional images, e.g. cross-sections, along various planes. Herein, a “three dimensional imaging data” can be used interchangeably with “3D data set.” A 3D data set can be used to generate cross-sectional images along various planes within the dimensions of the data set.

FIG. 1A illustrates a group of images. FIG. 1A shows an intra-op image 101 which can be captured and/or stored in memory during a surgical operation. FIG. 1A shows a matching image 201 which has a field of view which matches the field of view of the intra-op image.

The matching image 201 can be determined from image data 250 such as tomographic data (e.g. MRI data), and/or a 3D data set. The image data 250 can be encoded with spatial coordinate information, e.g. a coordinate system which allows for a cross-sectional image (e.g. one that may be a candidate for the matching image) to be generated in a given plane and/or region of the coordinate system (e.g. an image corresponding to a part of a plane of the coordinate space of the 3D data set). The plane can be selected by the user and/or determined algorithmically.

The image data 250 can be stored in memory and/or captured/acquired pre-operatively, in this example. The matching image 201 can be a slice and/or cross-section, e.g. through a 3D data set and/or tomographic data set. The matching image 201 can be the cross-sectional image and/or slice which corresponds to the plane of the image data set so that the matching image 201 has a field of view that matches the field of view of the intra-op image 101. For example, the matching image 201 is generated by constructing a cross-sectional image which passes through multiple adjacent 2D slices of a tomographic or 3D data set. It is convenient to be able to compare images from different sources.

During surgery, intra-operative images such as intra-op image 101 can be provided in real time. The intra-op image 101 may be acquired/captured by any one or more medical imaging techniques, e.g. reflectance and/or fluorescence microscopy. The intra-op image 101 can be, for example, a microscope image (including a white light or fluorescence image), a camera image, an endoscopic image, an ultrasound image, a magnetic resonance image, a computed tomography (CT) image, an optical coherence tomographic image, or an x-ray image.

In an example, a real-time microscope image is acquired during the surgical operation. Along with the real-time microscope image, it is possible for a surgeon to be able to view pre-op images, e.g. images taken before the surgical procedure. For example, an image can be constructed from pre-op tomographic data, e.g. magnetic resonance imaging (MRI) data. It can be particularly useful, during surgery, to have an image determined from a slice of MRI which matches the field of view of a real-time intra-op surgical image. For the surgeon or other medical professional, being provided with matching fields of view can ease the comparison of the real-time image with pre-op images. This may aid the medical professionals in planning and carrying out the surgical procedure.

In addition to providing a matching image from MRI data in real time (the MRI image matching the field of view of another intra-op image), during surgery, herein are disclosed methods and devices for providing matching images from different modalities and/or taken a different times (e.g. including pre-op, intra-op, and post-op images). A matching image(s) can be viewed post-operatively, e.g. along with the intraop image 101 captured during the operation. For example, after the surgical operation, it can be useful to compare images taken intra-operatively (intra-op images) and those taken pre-operatively (pre-op images). It can be useful to be able to compare post-op images as well.

When images and/or image data (e.g. tomographic data) are taken by multiple modalities and/or at different times, it can be challenging for a user such as a medical practitioner to efficiently compare images. It can be challenging for medical professionals to sort through imaging data from various sources and/or taken at various times to identify or generate images that aid intuitive comparison of surgical sites and the like. The methods and devices disclosed herein may facilitate easier comparison of images of surgical sites and the like. One of the challenges is matching the fields of view of images taken at different times and/or from different imaging modalities. In one example, 3D data, (e.g. tomographic data, such as MRI data), may be processed in order to provide a field of view which matches the field of view of an intra-op image (e.g. a 2D image) taken with a camera.

FIG. 1B illustrates a method of image processing. The method 190 includes selecting 191 an intra-op image 101 which is stored in memory. The intra-op image 101 has a field of view. The method 190 includes determining 192 a matching image 201 based on: selecting the matching image 201 from the memory, or constructing the matching image 201 from image data 250. The matching image 201 matches the field of view of the intra-op image 101.

FIG. 2A shows a plurality of intra-op images. A plurality of intra-op images 150 can be stored in memory. A surgeon, during a surgical procedure, may store a plurality of intra-op images 150, e.g. intra-op images 150 taken at different times and/or positions during the surgical operation. The intraop images 150 may be stored automatically and/or by user action such as triggering an image capture.

FIG. 2B shows an intraop image and associated parameters. Each intra-op image 101 can have stored parameters 101a, 101b, 101c which are associated with the intraop image 101. The parameters 101a, 101b, 101c may allow for determination of the field of view of the intraop image 101. For example, the parameters 101a, 101b, 101c can include any one or more of: a time stamp, working distance, a magnification, a position, orientation vector. The parameters 101a, 101b, 101c may be determined by sensors, user input, and/or algorithms.

In an example, when the intra-op image 101 is captured, the intra-op image 101 is stored in memory, and the matching image 201 is stored in memory. The matching image 201, which may be generated using image data 250 which was acquired pre-operatively, can be determined when the intra-op image is captured, e.g. such that the field of view of the matching image 201 matches the field of view of the intra-op image 101. The determination of the matching image 201 can be done in real time during the surgery, and the matching image 201 stored. It can be convenient for the matching image 201 to be stored, e.g. in association with the intra-op image 101 (which is also stored in this example). This approach may reduce later the computational burden of generating the matching image 201 from the image data 250.

For example, the image data 250 includes 3D data such as tomographic data. Constructing the matching image can include determining a plane of cross-section of the 3D and/or tomographic data. The determination and/or construction of the cross-section can be done post-operatively. For example, the construction can be based on the parameters 101a, 101b, 101c associated with the intra-op image 101. The parameters 101a, 101b, 101c can be determined and/or stored during the surgery, e.g. when the intra-op image is captured. The parameters 101a, 101b, 101c can include parameters for determining the field of view of the intraoperative image (e.g. working distance, magnification, orientation vector). The parameters 101a, 101b, 101c can be used to determine the cross-section of the image data 250 (which may be 3D data and/or tomographic data). Alternatively/additionally, it is possible to store the coordinates of the plane of the cross-section of the 3D data and/or tomographic data of the image data 250 that matches the field of view of the captured intraop image 101.

The parameters 101a, 101b, 101c can include coordinates of the plane of cross-section of the 3D and/or tomographic data that match the coordinates of the plane of the intraop image 101 that is stored in memory. This is one example of how the matching image 201 can be at least partially determined 192, e.g. by using parameters 101a, 101b, 101c associated with the intraop image 101. The parameters 101a, 101b, 101c may include coordinates of the plane of cross-section of the image data 250 which can at least partially provide the matching field of view. For example, the parameters 101a, 101b, 10c can be used for reducing the computational cost of determining the matching image 201.

The stored parameters 101a, 101b, 101c can include a position and an orientation for providing the field of view of the intraop image 101 and the matching image 201. For example, the stored parameters 101a, 101b, 101c include also an areal range of the cross-sectional plane of the image data the corresponds to the field of view of the intraop image 101. For example, when the intraop image 101 is a high magnification microscopic image, the field of view of the intraop image 101 may only extend a few millimeters. The cross-sectional plane of the image data 250 may extend along a plane much bigger than that. Therefore, the parameters 101a, 101b, 101c may include a magnification, working distance, and/or focal distance which can provide a way to compute an extent of the field of view, e.g. the areal range of the field of view. To determine the matching field of view, the parameters 101a, 101b, 101c can include data for identifying the cross-sectional plane of the image data 250, a center of the field of view within the appropriate cross-sectional plane, and the extent of the field of view (e.g. the areal range of the field of view).

The stored parameters 101a, 101b, 101c can include user input. For example, user input can be used to estimate the position of a particular feature or field of view. In an example, a routine surgical procedure may have a standard and/or commonly used optical arrangement for acquiring the intraop images 101, 102. For example, many eye operations have a standard optical setup such that the intraop images are acquired within a narrow range of orientations with respect to the patient. In such a case, user input can provided at least an initial estimate of the field of view and/or perspective of the intraop image(s) 101, 102. The field of view of the intraop image(s) 101, 102 may correspond to a frequently used surgical perspective. For example, routine surgeries may use routine placement of cameras and/or other imaging equipment, such that there may be a standardized and/or expected field of view. The stored parameters 101a, 101b, 101c can include identification of a defined and/or estimated perspective, field of view, orientation, and/or position.

In another example, an endoscopic image may be captured as the intraop image, and the stored parameters 101a, 101b, 101c can include an estimated position, perspective, and/or field of view of the field of view. Positions, perspectives, and the like can be determined by sensors that can provide the position and/or orientation of the camera. Sensors may be internal and/or external to the camera device.

Additional user input that can be stored in the stored parameters 101a, 101b, 101c can include an estimated size of a feature, such as a diameter of a feature. In an example, a lesion's size can be estimated and the surgeon can input the estimation.

FIG. 3 illustrates a group of images. The group 300 includes an intraop image 101 and a matching image 201. The group also includes a second intraop image 102 and a second matching image 302. The first matching image 201 has a field of view that matches the field of view of the first intraop image 101. The second matching image 302 has a field of view that matches the field of view of the second intraop image 102. It is also possible that the first and second intraop images 101, 102 have the same field of view, and that the matching images 201, 302 each have fields of view that match.

For example, it may be useful to capture intraop images 101, 102 before and after a resection or other procedure performed during the surgery. It can be useful to have the intraop images 101, 102 at the same position to aid comparison between the intraop images 101, 102. The intraop images 101, 102 can be compared to each other, e.g. by medical professionals and/or trainees after the surgery. Matching images 201, 302 can also be compared. Having the same perspective and/or field of view for the images 101, 102, 201, 302 can facilitate and ease comparison, particularly when there may be significant changes to the anatomical structure due to surgical activities at the surgical site within the field of view.

A surgeon may capture a first intraop image 101 and second intraop image 102 at the same or different regions of the patient. FIG. 3 is an example in which the intraop images 101, 102 are taken from the same place at different times, e.g. before and after resection. In an example, a first field of view is captured in an intraop image, then a second field of view from a different perspective is captured. Intraop images from different fields of view may also be matched with respective matching fields of view from stored image data (e.g. yielding two pairs of intraop and matching images).

Multiple intraop images 101, 102 may be captured, at same or different fields of view. With each captured intraop image 101, 102, it is possible to also store a corresponding matching image 201 particularly when the image data 250 for generating the matching images 201, 302 has been processed to determine the matching image 201, 302. Alternatively/additionally, parameters 101a, 101b, 101c can be stored with each intra-op image. The parameters 101a, 101b, 101c may provide information to allow reconstitutions and/or generation of the corresponding matching images 201, 302 from image data 250, e.g. at a later time.

Matching images 201, 302 can be determined from image data that is taken pre-operatively, intraoperatively (e.g. when multiple imaging modalities are hosted in the surgical suite), and/or post-operatively. The example of FIG. 3 shows a situation where the first matching image 201 can come from image data 250 taken pre-operatively, and the second matching image 302 can come from image data 350 (see FIG. 4) that is taken post-operatively. The fields of view of the images 101, 102, 201, 302 may be matching, as shown in FIG. 3. It can be useful to compare images from pre-, intra-, and/or post-op images in order to determine outcomes of a surgical intervention, for example.

The image data 250 can include a coordinate system, explicitly or implicitly. Image data can be a three dimensional data set, such as a tomographic data set, plurality of slices, and/or cross-sectional images taken along a direction so as to generate a three-dimensional data set or tomographic data set. It is possible to construct or reconstruct the matching image 201 from one a three dimensional data set, e.g. by determining the plane of cross-section that provides the same field of view as the corresponding intraop image 101. The parameters 101a, 101b, 101c associated with the intraop image 101 (or that of any intraop image such as second intraop image 102) can be used to determine the plane of cross-section to be taken. The parameters 101a, 101b, 101c may be related to the coordinates of the image data 250 that produces the matching image 201 in real time during the surgery. Alternatively/additionally, the parameters 101a, 101b, 101c can be used in combination with other approaches (e.g. computer vision algorithms such as including feature recognition, such as edge recognition) to determine the matching image 201.

For example, there may be real-time matching of the field of view of the intraop image 101 with a matching image 201, which can be determined from the image data 250 in real time. The real-time matching may operate, for example, by determining the coordinates (e.g. position and orientation) of the detector that captures intraop images in real-time. The determination of coordinates may be done by calibrating the position of the intraop image detector(s) with respect to fiducial marks on an optical table and/or the patient. For example, a second camera may be used to determine the relative positions of the fiducial markers and the intraop image detector. When an intraop image 101 is stored, for example, the relative positions of the intraop image detector and the fiducial marks can be used to determine the position and orientation of the intraop image detector; the possible coordinates of the plane of the matching image 201 can be constrained by determining the position and orientation of the intraop image detector. Further constraints can be determined from the magnification and/or working distance of the intraop camera, for example.

The parameters 101a, 101b, 101c (which may include any combination of the position and orientation of the intraop image detector, and the acquisition parameters for the intraop image 101 such as magnification and working distance) can be stored. The parameters 101a, 101b, 101c can be used subsequently, e.g. after the surgery, in any subsequent determination of the matching image 201 that has a matching field of view as the intraop image 101. The subsequent determination of the matching image 201 can be done, for example, without using the fiducial marks for calibrating the position of the intraop image detector. Alternatively/additionally, the fiducial marks are used during the surgery for calibration of the position and/or orientation of the intraop image detector; and the position and/or orientation of the intraop image detector is stored as one or more of the parameters 101a, 101b, 101c for subsequent determination of the matching image 201 (e.g. possibly in combination with other approaches as described herein).

Multiple intraop images 101, 102 can be stored. A second intraop image 102 can be stored during the surgical procedure, e.g. as a second of a plurality of intraop images. The second intraop image 102 can have associated parameters stored, e.g. in metadata, that will allow determination of the field of view of the second intraop image 102, e.g. to be used in the determination of a matching image 302 from image data from one or more sets of image data.

In an example, the acquisition parameters of the first intraop image 101 are the same as the second intraop image 102. This can be useful to compare the intraop images 101, 102. In the example of FIG. 3, it is possible that a resection is performed after the first intraop image 101 is captured and before the second 102 is captured. Additionally, it is possible that the first matching image 201 is a pre-op image (e.g. an image showing a lesion), and the second matching image 302 is a post-op image (e.g. an image showing the region of the lesion after the resection and after the surgical procedure is finished). Acquisition parameters can include magnification, working distance, filter settings, lamp brightness, detector position, and/or detector orientation.

FIG. 4 illustrates a memory device. The memory 400 can store a group of images. The group of images can include one or more intraop images 150, a first set of image data 250, and a second set of image data 350. For example, the first and second sets 250, 350 of image data can be pre- and post-op data sets, respectively. The memory 400 can be one or more memory devices, including possibly cloud storage and/or remote storage device(s) which can be in communication with an image processing device.

When an intraop image is captured, e.g. by a user triggering a capture of a real-time image during a surgical procedure, the captured image can be stored as one of the plurality of intraop images 150 stored in the memory 400. It is possible for the memory 400 to also store any of the matching images of the intraop images (e.g. matching image 201 which matches the field of view of the intraop image 101, and/or matching image 302 which matches the field of view of the intraop image 102). Alternatively/additionally, it is possible to generate (or regenerate as the case may be) the matching images 201, 302 from the image data 250, 350 which is stored in the memory 400. For example, an intraop image 101 (which may include metadata and/or associated stored parameters 101a, 101b, 101c) can be used to determine the matching image 201, which can be generated from the image data 250. In another example, the intraop image 102 (which may include metadata and/or associated stored parameters) can be used to determine the matching image 302, which can be generated from the image data 350.

FIG. 5 illustrates a surgical imaging system. The surgical imaging system 500 may include or may be a computer device (e.g. personal computer, laptop, tablet computer or mobile phone) with the one or more processors 510 and one or more storage devices 520 located in the computer device or the system 500 may be a distributed computing system (e.g. cloud computing system with the one or more processors 510 and one or more storage devices 520 distributed at various locations, for example, at a local client and one or more remote server farms and/or data centers). The system 500 may include a data processing system that includes a system bus to couple the various components of the system 500. The system bus may provide communication links among the various components of the system 500 and may be implemented as a single bus, as a combination of busses, or in any other suitable manner. An electronic assembly may be coupled to the system bus. The electronic assembly may include any circuit or combination of circuits. In one embodiment, the electronic assembly includes a processor which can be of any type. As used herein, processor may mean any type of computational circuit, such as but not limited to a microprocessor, a microcontroller, a complex instruction set computing (CISC) microprocessor, a reduced instruction set computing (RISC) microprocessor, a very long instruction word (VLIW) microprocessor, a graphics processor, a digital signal processor (DSP), multiple core processor, a field programmable gate array (FPGA) of the microscope or a microscope component (e.g. camera) or any other type of processor or processing circuit. Other types of circuits that may be included in electronic assembly may be a custom circuit, an application-specific integrated circuit (ASIC), or the like, such as, for example, one or more circuits (such as a communication circuit) for use in wireless devices like mobile telephones, tablet computers, laptop computers, two-way radios, and similar electronic systems. The system 500 includes one or more storage devices 520, which in turn may include one or more memory elements suitable to the particular application, such as a main memory in the form of random access memory (RAM), one or more hard drives, and/or one or more drives that handle removable media such as compact disks (CD), flash memory cards, digital video disk (DVD), and the like. The system 500 may also include a display device, one or more speakers, and a keyboard and/or controller, which can include a mouse, trackball, touch screen, voice-recognition device, or any other device that permits a system user to input information into and receive information from the system 500.

Additionally, the system 500 may include a microscope connected to a computer device or a distributed computing system. The microscope may be configured to generate the biology-related image-based input training data 104 by taking an image from a biological specimen.

The microscope may be a light microscope (e.g. diffraction limited or sub-diffraction limit microscope as, for example, a super-resolution microscope or nanoscope). The microscope may be a stand-alone microscope or a microscope system with attached components (e.g. confocal scanners, additional cameras, lasers, climate chambers, automated loading mechanisms, liquid handling systems, optical components attached, like additional multiphoton light paths, lightsheet imaging, optical tweezers and more). Other image sources may be used as well as long as they can take images of objects which are related to biological sequences (e.g. proteins, nucleic acids, lipids). For example, a microscope according to an embodiment described above or below may enable deep discovery microscopy.

FIG. 5 illustrates an imaging processing device 501 coupled to a surgical imaging device 550. An image processing device 501 may include a processor 510, e.g. a computer processor. The imaging device (e.g. surgical imaging system 500) can be communicatively coupled to the memory 400 and/or include an internally located memory storage device 520 which may be part of the memory 400 that stores images 101, 102, 201, 302, and/or image data 250, 350. The device can have a display 530. The image processing device 501 can be coupled to a surgical imaging device 550 such as a microscope. The imaging processing device 501 and surgical imaging device 550 can form a surgical imaging system 500.

The processor 510 can be used to perform the methods described herein, such as methods 190 of imaging processing, determining field of view, orientations and/or positions of fields of view of optical devices such as detector 570. FIG. 5 shows a virtual field of view 580. The virtual field of view 580 is movable with the surgical instrument 550. Herein the field of view refers to the captured field of view of the patient/tissue, e.g. during surgery or imaging process. The field of view of the intraop image 101, 102 can be determined by the position/orientation of the patient/tissue with respect to the virtual field of view 580 shown in FIG. 5.

For example, during surgery, the image processing device 501 can be communicatively coupled to a surgical instrument 550 that can include the intraop image detector (e.g. a camera). As shown in FIG. 5, the surgical instrument 550 can be a microscope, e.g. a surgical microscope. The surgical instrument may be another type of imaging device such as an ultrasound device, optical coherence tomography device, or camera.

The image processing device 501 can include a memory storage device 520 and/or be coupled to and memory 400. Image data 250 can be accessed in local and/or remote memory, for example. The processor 510 (which can have multiple cores and/or multiple processors) can be used for image processing. The image processing device 501 can determine the matching image 201 from the image data 250 such that the matching image 201 has a field of view that matches the field of view of the intra-op image 101. The anatomical features visible in the intraop image can be in registry with the anatomical features visible in the matching image 201, when the fields of view match. For example, a superposition of an intraop image 101 and a matching image 201 would have features of the fields of view of the images 101, 201 at the same positions, e.g. such that the features overlap in registry.

FIG. 5 also shows an intraop image detector 570 which may be a camera for collecting light from the surgical site. The image detector 570 can be oriented along an optical axis which is coaxial with the vector 560 shown in FIG. 5.

FIG. 5 shows a vector 560 which may be used as an orientation vector which can be sensed and recorded, e.g. with the stored intraop image(s) 101, 102. The vector may include positional and directional information, e.g. for determining the field of view of the microscope. The vector 560, when sensed and recorded, may provide the position and/or orientation for at least partially determining the field of view of the captured intraop image(s) 101, 102. The working distance, focal distance, magnification, and/or other optical parameters may also be recorded for determination of the position/orientation of the field of view of the intraop image(s) 101, 102, e.g. by determination of the position/orientation of the image detector 570 and/or the optical axis thereof. The matching image 201, 302 can be determined such that the field of view of the matching image 201, 302 matches the field of view of the intraop image 101, 102.

Sensors for determining the position/orientation of the intraop image(s) 101, 102 may be employed. For example, accelerometer(s) can determine motions of the intraop image detector 570. External 3D camera(s) can be employed to track/record positions of the imaging apparatus, microscope, image detector 570, and/or the optical axis (e.g. vector 560) thereof. Electromechanical and/or optomechanical sensors can be employed to record the relative positions of the booms, arms and any other movable mechanical components for positioning the surgical imaging device 550 and/or field of view thereof.

In an example, during surgery, the intra-op image 101 is captured, and the matching image 201 is determined, e.g. by construction of the matching image 201 from image data 250. It is possible to store the intra-op image 101 and the matching image 201 in memory 400 such as in the memory device 520. Additional intraop images (e.g. second intraop image 102) can be stored. Matching images which are constructed from image data 250 such that the matching images each have fields of view that match the fields of view of corresponding intraop images 101, 102 can also be stored. For example, the first intraop image has a first field of view which is matched by the field of view of the first matching image; and the second intraop image has a second field of view which is matched by the field of view of the second matching image, or all four can have the same field of view.

In an example, image data 250 is image data from pre-operative imaging, such as a magnetic resonance imaging (MRI), computed tomography (CT), ultrasound, positron emission tomography (PET), nuclear medicine imaging, x-ray, single-photon emission CT, or other imaging methods. Image data 250 can provide a three dimensional image of the tissue, surgical site, and/or patient. Alternatively/additionally, the image data 250 can include scanning data that may provide a three dimensional representation of a patient's anatomy, and/or may be usable to generate two-dimensional images along a variable cross-sectional plane. The image data 250 can be processed to provide cross sections from variable perspectives and/or variable fields of view.

In an example, the matching field of view of the matching image 201, 302 can be determined at least partially by computer vision algorithms, a feature recognition algorithm (e.g. object identification, object recognition, and/or object classification), a matching algorithm, and/or an image comparison algorithm. For example, the intraop image 101 can be processed for edge detection. Alternatively/additionally, patient vasculature can be used for image recognition and/or alignment and matching of the fields of view. Examples of edge detection may utilize Canny edge detection, Canny-Deriche detection, differential edge detection, and/or phase stretch transform, for example. The intraop image 101 and/or an edge enhanced version thereof can be used to determine similarities and/or differences between the intraop image 101 and slices and/or cross-sections of the image data 250. An algorithm may minimize calculated differences and/or maximize calculated similarities to determine a slice/cross-section of the image data 250 that matches the intraop image 101. For example, mean square error can be minimized and/or structural similarity index can be maximized.

It is recognized that the computational burden of identifying the cross-section of the image data 250 which has the most similarity (and/or least mean square error) with the intraop image 101 can be high.

There are numerous strategies to reduce the computational burden of determining the matching image 201, 302 which matches the respective field of view of the respective intraop images 101, 102.

The computation may rank the likelihood of multiple possible cross-sections of the image data 250 to determine which cross-sections have greater likelihood to have a matching field of view to that of the intraop image 101. The ranking may be based on least squares, for example.

The determination of the matching image 201 can be aided by user input and/or stored parameters 101a, 101b, 101c.

It is recognized that user input may be used to aid in identifying the edge of a feature, e.g. the surgeon may input a drawn edge that is traced over the boundary of a feature, e.g. a tumor or part of the patient vasculature. The user input can be used to define the feature and/or as an initial estimate of the feature which may be refined by a computer vision algorithm, a feature recognition algorithm (e.g. object identification, object recognition, and/or object classification, for example.

The user input, which can be associated with the intraop image and/or the image data, can also include a label. The label may aid in matching/comparing the user highlighted feature (of the intraop image 101, for example) with the corresponding feature from the other image and/or image data (the image data 250, for example). A label may also include a reference or note, such as reference to a tissue sample taken from the field of view of the captured intraop image 101. Medical practitioners can find it convenient to be able to compare an image of a surgical site with any results of tissue analysis of samples from various positions in the surgical site. For example, histology tests of the tissue sample can be performed, and the histology results linked to the intraop image 101.

For example, it is possible to constrain the cross-sections of the image data 250, 350 that are searched as candidates for having a matching field of view. For example, the surgery may be performed with the surgical field of view being at a known range of orientations with respect to the detector/camera/microscope used to capture the intraop image(s) 101, 102. For example, the intraop image(s) 101, 102 is expected or known to be in a range of possible orientations with respect to the coordinate system of the patient and/or image data 250, 350. In determining the matching image 201, 302, the possible cross-sections of the image data 250, 350 can be reasonably constrained, e.g. to exclude orientations/cross-sections that are from a perspective outside of the known or expected range of perspectives. Such constraints can reduce the computational burden, e.g. by reducing the space of possible cross-sectional candidates, and may increase the accuracy of the determination of the matching image(s) 201, 302. Such constraints may also aid in determining an initial guess of the matching image 201, 302 and/or expected range of deviation from the initial guess, such that the algorithm can find the matching image 201, 302 more rapidly.

The determination of the matching image 201, 302, may be based at least partially on constraining the possible cross-sections of the image data 250 which are to be compared to the intraop image(s) 101, 102. The constraints may be based on expected and/or known orientations of the detector/camera/microscope used to capture the intraop image(s) 101, 102. Such constraints may be stored, e.g. associated with the intraop images(s) (e.g. as parameters 101a, 101b, 101c). Such constraints may be based on user input.

For example, a medical practitioner, e.g. one in the surgical suite, can input an expected and/or known range of orientations of the detector/camera/microscope that captures the intraoop image(s). Alternatively/additionally, the expected range of orientations can be determined based on an input related to the type of surgery to be performed. For example, eye surgeries and other types of surgeries may have an expected range of positions and/or orientations of the intraop image detector/camera/microscope which can be used to constrain the search of cross-sections of the image data 250 in determining the matching image(s) 201, 302.

In another example, a user such as a medical practitioner can input parameters that are stored to aid in determining the matching image(s) 201, 302, such as by feature recognition. For example, entering an initial guess of position of a feature and/or field of view of the intraop image 101 can reduce the computational burden and speed up the determination of the matching image. For example, a user may input an identification of a feature as being a known anatomical structure, e.g. identifying a field of view of the inatraop image 101, 102 as including a known anatomical structure may aid in reducing the computational burden on determining the matching image 201, 302, e.g. by constraining the range of possible cross-sections and/or regions of the image data 250, 350 for which to search for the matching image 201, 302. Alternatively/additionally, a user can input an estimated size and/or position of a feature of interest, e.g. the size/position of a lesion.

In another example, multiple intraop images 101, 102 may be taken to have the same position and orientation to provide the same field of view (possible with some change in features due to resection or the like). It is possible that one intraop image, e.g. second intraop image 102, refers to another intraop image, e.g. first intraop image 101, as a basis for the determination of the appropriate cross-section, slice, and/or region of the image data 250, 350. Alternatively/additionally, an intraop image 102 may refer to the matching image 201 which is determined from another intraop image 101 in order to determine the cross-sectional plane, slice, and/or region of the image data 250, 350. This can be the case when, for example, a subsequent intraop image, e.g. second intraop image 102, is taken at the same field of view as a previous intraop image, e.g. first intraop image 101. Such a procedure can be useful, such as to record the appearance of the surgical site before and after resection, for example.

Medical professionals can find it convenient to have a real-time intra-op image 101 and a matching image 201 which has the same field of view. It is possible to overlay or superimpose images that have the same field of view, which may aid the medical professional in interpreting the images. It is also convenient to have the intra-op image 101 and the matching image 201 provided after the surgery. The methods, devices, systems, and/or programs described herein may aid in providing, to medical professionals and/or students, convenient medically relevant images for comparison. This can aid in record keeping, tracking patient outcomes, and/or for teaching medical professionals/students.

The methods, devices, systems, and/or programs described herein can be employed, at least partially, after a surgery is complete. There remains considerable interest in providing medical practitioners, outside of the surgical suite, ways of managing images from multiple sources which may include images acquired from imaging apparatuses within and outside of the surgical suite. Furthermore, current methods of providing images (constructed from 3D image data) having matching fields of view to images captured interaoperatively may rely on particular pre-op calibrations of sensors and/or fiducial marker(s) which may not be possible post-operatively. The methods, devices, systems, and/or programs described herein may aid medical professionals, in a non-surgical suite environment that may be without sensors and/or fiducial markers for calibration, by providing images with matching fields of view, the images (and/or image data) being acquired from multiple modalities and/or at different times.

FIG. 6 illustrates a block diagram of a surgical imaging system. A microscope can include a computing unit which is communicatively coupled to a microscope device controller (MDC), e.g. through RS232 cabling. A controller area network bus (CAN-BUS) can communicatively couple the microscope to an image guided surgery system (IGS System). DVI-I, as shown in FIG. 6 can be an overlay video input Digital Visual Interface. SDI, as shown in FIG. 6, can be a video output serial digital interface which can be coupled by BNC (Bayonet Neill-Concelman) connectors. The microscope and IGS system can be synchronized, e.g. having synchronized clocks.

In acquisition/capture, an image can be taken by the Computing Unit and stored, optionally with a timestamp. The timestamp can be stored and/or sent to the MDC. Settings (e.g. acquisition parameters) can be determined/sensed/acquired by the Computing Unit, e.g. simultaneously (e.g. as step 1a) and stored, optionally with the timestamp. Settings can be for example working distance, illumination intensity, magnification, bright care status, brakes status, FL thresholds etc. The settings (which can be stored, e.g. in association with the stored captured intraop image by the microscope) can be for determining the matching image of image data which has a matching field of view as the captured image.

In the embodiment of FIG. 6, it is possible that a trigger, ping, and/or timestamp is sent to the IGS system (e.g. by the CAN-BUS, which can be a one-way communication path, from MDC to IGS System) to generate the matching image. The matching image may be generated by the IGS System and communicated to the Computing Unit via the DVI-I, during the surgery, for example. The Computing Unit can possibly cause the storing of the generated matching image (and the corresponding captured intraop image), e.g. for later analysis by a medical practitioner.

FIG. 7 illustrates a block diagram of a surgical imaging system. The system of FIG. 7 is similar to that of FIG. 6. There may be 2-way communicative coupling of the Computing Unit and the IGS System, for example. FIG. 7 shows an ethernet connection that communicatively couples the computing unit and the IGS System. The ethernet connection may, for example, allow the Computing Unit to receive (and possibly store) parameters acquired from the IGS System that allow for the determination of the matching image. For example, the acquired parameters are coordinates of the cross-section of the image data that correspond to the slice which includes the matching image and the matching field of view as the intraop image. Such coordinates can be stored, e.g. in association with the intraop image, as parameters (e.g. in meta-data of the intraop image). The matching image can be subsequently re-determined (e.g. post-operatively) using the coordinates. In the case of Ethernet communication, both devices can even exchange images (and the microscope can still send a ping or timestamp).

It is noted that additional information can be associated with the recorded images, such as illumination and camera settings, white light image, fluorescence image, time stamp, labels (e.g. for linking the stored intraop images with tissue samples which may be analyzed post-operatively). Alternatively/additionally, patient data can be imported (e.g. from MWL [DICOM Modality Worklist], which can be optionally anonymized.

Data can be stored in Picture Archiving Communication System (PACS) format, for example. Archiving can be done directly to the DICOM node and/or through IGS.

Stored images can be loaded into a post-op analysis application, e.g. of an image processing device (such as may be part of a computer station). The image processing device can be communicatively coupled to a hospital information system (HIS) and/or radiology information system (RIS). The images can be stored compatibly with a Picture Archiving Communication System (PACS).

Some or all of the method steps described herein may be executed by (or using) a hardware apparatus, like for example, a processor, a microprocessor, a programmable computer or an electronic circuit.

The methods described herein can be implemented in hardware or in software. The implementation can be performed using a non-transitory storage medium such as a digital storage medium, for example a floppy disc, a DVD, a Blu-Ray, a CD, a ROM, a PROM, and EPROM, an EEPROM or a FLASH memory, having electronically readable control signals stored thereon, which cooperate (or are capable of cooperating) with a programmable computer system such that the respective method is performed. The digital storage medium may be computer readable.

Some embodiments according to the invention include a data carrier having electronically readable control signals, which are capable of cooperating with a programmable computer system, such that one of the methods described herein is performed.

Embodiments described herein can be implemented as a computer program product with a program code, the program code being operative for performing one of the methods when the computer program product runs on a computer. The program code may, for example, be stored on a machine readable carrier.

Other embodiments include the computer program for performing one of the methods described herein, stored on a machine readable carrier.

Herein is disclosed a computer program having a program code for performing the methods described herein, when the computer program runs on a computer.

Herein is disclosed a storage medium (or a data carrier, or a computer-readable medium) comprising, stored thereon, the computer program for performing the methods described herein when it is performed by a processor. Herein is disclosed an apparatus as described herein comprising a processor and the storage medium for executing the methods described herein.

Herein is disclosed a data stream or a sequence of signals representing the computer program for performing one of the methods described herein. The data stream or the sequence of signals may, for example, be configured to be transferred via a data communication connection, for example, via the internet.

Herein is disclosed a processing means, for example, a computer or a programmable logic device, configured to, or adapted to, perform the methods described herein.

Herein is disclosed a computer having installed thereon the computer program for performing the methods described herein.

In some embodiments, a programmable logic device (for example, a field programmable gate array) may be used to perform some or all of the functionalities of the methods described herein. In some embodiments, a field programmable gate array may cooperate with a microprocessor in order to perform one of the methods described herein. The methods described herein are preferably performed by any hardware apparatus.

Although some aspects have been described in the context of an apparatus, it is clear that these aspects also represent a description of the corresponding method. Analogously, aspects described in the context of a method step also represent a description of a corresponding apparatus.

List of reference signs

| | |
|---|---|
| 101 | intraop image |
| 101a, 101b, | stored parameters |
| 101c | |
| 102 | second intraop image |
| 150 | plurality of intraop images |
| 190 | method of image processing |
| 191 | selecting an intraop image |
| 192 | determining a matching |
| | image |
| 201 | matching image |
| 250 | image data |
| 300 | group of images |
| 302 | second matching image |
| 350 | image data |
| 400 | memory |
| 500 | surgical imaging system |
| 501 | image processing device |
| 510 | processor |
| 520 | memory storage device |
| 530 | display |
| 550 | surgical imaging device |
| 560 | vector |
| 570 | detector |
| 580 | field of view |

1. An image processing device, configured for:
selecting an intra-op image which is stored in a memory, wherein the intra-op image has a field of view; and
determining a matching image based on:
selecting the matching image from the memory, or
constructing the matching image from image data; wherein the matching image matches the field of view of the intra-op image.

2. The image processing device of claim 1, further configured for selecting the intra-op image from a plurality of intra-op images stored in the memory.

3. The image processing device of claim 1, further configured for
determining a second matching image, wherein the second matching image matches the field of view of the intra-op image, and wherein the matching image is a pre-op image, and the second matching image is a post-op image.

4. The image processing device of claim 1, wherein
the intra-op image is an image captured by a surgical imaging apparatus.

5. The image processing device of claim 4, wherein
the surgical imaging apparatus is a microscope, infrared microscope, optical coherence tomography device, photoacoustic imaging device, ultrasound imaging device, magnetic resonance imaging device, or computed tomography device.

6. The image processing device of claim 1, further configured for:
selecting a second intra-op image which is stored in memory, wherein
the second intra-op image matches the field of view of the intra-op image; and wherein the second intra-op image is automatically selected when the intra-op image is selected.

7. The image processing device of claim 6, wherein the second intra-op image is captured at a plurality of acquisition parameters, and the first intra-op image is captured at the plurality of acquisition parameters.

8. The image processing device of claim 1, wherein
the image data includes three dimensional imaging data, and
the image processing device is further configured for:
constructing the matching image by
determining a plane of cross-section of the three dimensional imaging data based on:
a plurality of stored parameters associated with the intra-op image.

9. The image processing device of claim 8, wherein
the plurality of stored parameters includes at least one of: a user input, a position of a detector, an orientation of the detector, a plurality of reference positions, a magnification, a focal length, or a working distance.

10. The image processing device of claim 1, wherein
the image processing device is further configured for:
constructing the matching image by an algorithm which includes edge recognition.

11. The image processing device of claim 1, wherein
the image processing device is configured for:
generating the matching image by sending a ping or timestamp to an image guided surgery system, and obtaining the matching image from the image guided surgery system.

12. The image processing device of claim 1, wherein
the image processing device is configured for:
bidirectionally communicating with an image guided surgery system, and
obtaining at least one of the matching image and parameters of the image guided surgery system that allow for a determination of the matching image from the image guided surgery system.

13. A surgical imaging device, configured for:
capturing an intraop image,
determining a matching image based on constructing the matching image from image data;
storing the intraop image, and
storing the matching image or a plurality of acquisition parameters; wherein
the acquisition parameters are for the determination of the matching image from the image data; wherein
the matching image matches the field of view of the intra-op image.

14. The surgical imaging device of claim 13, further comprising:
a detector for capturing the intraop image; wherein
the acquisition parameters are at least one of: working distance, magnification, focal length, position of the detector of the intraop image, orientation of the detector, coordinates of a cross-section of the image data which includes the matching field of view, or user input.

15. The surgical imaging device of claim 13, wherein
the surgical imaging device is configured for:
obtaining parameters of an image guided surgery system that allow for a determination of the matching image from the image guided surgery system, and determining the matching image based on the parameters of the image guided surgery system.

16. A method of image processing, comprising
selecting an intra-op image which is stored in memory, wherein
the intra-op image has a field of view; and
determining a matching image based on:
selecting the matching image from the memory, or
constructing the matching image from image data; wherein
the matching image matches the field of view of the intra-op image.

17. The method of claim 16, wherein
the intra-op image is selected by a user.

18. The method of claim 17, further comprising:
determining a plurality of parameters during a surgery,
capturing the intra-op image during the surgery, and
storing the plurality of parameters in association with the intra-op image.

19. The method of claim 18, wherein
the stored parameters include a position and an orientation for providing the field of view of the intra-op image.

20. The method of claim 16, wherein
constructing the matching image includes
recognizing edge features.

21. The method of claim 16, wherein
the matching image is:
a pre-op image or a post-op image.

22. The method of claim 16, further comprising:
determining a second matching image which is a post-op image, wherein
the matching image is a pre-op image.

23. The method of claim 16, further comprising:
selecting a second intraop image, and
determining a second matching image based on:
selecting the matching image from the memory, or
constructing the matching image from image data; wherein
the matching image matches the field of view of the second intra-op image.

24. The method of claim 23, further comprising:
determining a label for the captured image by user input, and
storing the label; and
determining a second label for the second captured image by user input, and
storing the second label.

25-26. (canceled)

27. A non-transitory, computer-readable medium comprising a program code that, when the program code is executed on a processor, a computer, or a programmable hardware component, causes the processor, computer, or programmable hardware component to perform the method of claim 16.
